(19) United States
(12) Patent Application Publication
(10) Pub. No.: US 2023/0170100 A1
(43) Pub. Date: Jun. 1, 2023

(54) MEDICAL DATA PROCESSING METHOD AND SYSTEM

(71) Applicant: Haoyu CHEN, Wenzhou City (CN)

(72) Inventor: Haoyu CHEN, Wenzhou City (CN)

(73) Assignee: Haoyu CHEN, Wenzhou City (CN)

(21) Appl. No.: 17/468,958

(22) Filed: Sep. 8, 2021

(30) Foreign Application Priority Data
Aug. 17, 2021 (CN) 202110941538.2

(51) Int. Cl. G16H 70/60; G16H 10/20

(52) U.S. Cl. CPC G16H 70/60; G16H 10/20

(57) ABSTRACT

Disclosed is a medical data processing method and system. The method comprises: acquiring pathogenic site information of a patient by means of a preset first question answer mode and extracting a keyword of the pathogenic site information; acquiring symptom information of the patient by means of a preset second question answer mode and extracting a keyword of the symptom information; performing case matching according to the keyword of the pathogenic site information and the keyword of the symptom information; and taking out a case with a highest matching degree from a case library, sending the case as a reference case to a doctor, and conducting registration for the patient automatically.

### TECHNICAL FIELD

The present invention relates to the technical field of medical information processing, in particular to a medical data processing method and system.

### BACKGROUND

When a patient sees a doctor, a long-time inquiry is usually needed, and therefore, the efficiency is relatively low. It is quite short in medical resource at present, and long-time repeated inquiry will lead to waste of medical resource. However, same disease is almost similar in symptom. If symptoms of the patient can be acquired in advance, the closest disease information is acquired according to a symptom characteristic and is sent to the doctor, and meanwhile, registration is conducted on the patient automatically, a lot of diagnostic time can be saved.

Thus, how to provide a method of diagnosing disease quickly is the problem demanding prompt solution by those skilled in the art.

### SUMMARY

The embodiment of the application provides a medical data processing method and system to solve the problem that the medical diagnosis time is too long.

In the first aspect, the application provides a medical data processing method. The method includes:
acquiring pathogenic site information of a patient by means of a preset first question answer mode and extracting a keyword of the pathogenic site information;
acquiring symptom information of the patient by means of a preset second question answer mode and extracting a keyword of the symptom information;
performing case matching according to the keyword of the pathogenic site information and the keyword of the symptom information; and
taking out a case with the highest matching degree from a case library, sending the case as a reference case to a doctor, and conducting registration for the patient automatically.

In an embodiment, after extracting a keyword of the pathogenic site information, the method further includes:
acquiring a pathogenic site of the patient according to the keyword of the pathogenic site information.

In an embodiment, before acquiring symptom information of the patient by means of a preset second question answer mode, and extracting a keyword of the symptom information, the method further includes:
selecting a corresponding second question answer mode according to the pathogenic site of the patient.

In an embodiment, before matching the keyword with the case in the case library, the method further includes:
classifying the case in the case library according to the pathogenic site and extracting a pathogenic symptom keyword as a case tag and storing the pathogenic symptom keyword.

In an embodiment, taking out a case with the highest matching degree from a case library includes:
matching the keyword of the symptom information with the case tag, and taking out the case corresponding to the case tag with the highest matching degree from the case library.

In an embodiment, the preset first question answer mode and the preset second question answer mode are question answer rules formulated by a physician or a specialist.

In the second aspect, the application further provides a medical data processing system. The system includes:
a first question answer module configured to acquire pathogenic site information of a patient by means of a preset first question answer mode, and extract a keyword of the pathogenic site information;
a second question answer module configured to acquire symptom information of the patient by means of a preset second question answer mode, and extract a keyword of the symptom information;
a matching module configured to perform case matching according to the keyword of the pathogenic site information and the keyword of the symptom information; and
a case acquisition module configured to take out a case with the highest matching degree from a case library, send the case as a reference case to a doctor, and conduct registration for the patient automatically.

In a third aspect, the application further provides a computer device, comprising a memory, a processor and a computer program stored in the memory and capable of running on the processor, wherein the processor realizes the medical data processing method as claimed in any one in the first aspect when executing the computer program.

In a fourth aspect, the application further provide a computer readable storage medium, having the computer program stored thereon, wherein the medical data processing method as claimed in any one in the first aspect is executed when the computer program is executed by the processor.

According to a medical data processing method and system, the method includes: acquiring pathogenic site information of a patient by means of a preset first question answer mode, and extracting a keyword of the pathogenic site information; acquiring symptom information of the patient by means of a preset second question answer mode, and extracting a keyword of the symptom information; performing case matching according to the keyword of the pathogenic site information and the keyword of the symptom information; and taking out a case with the highest matching degree from a case library, sending the case as a reference case to a doctor, and conducting registration for the patient automatically. According to the application, similar cases in the case library are acquired automatically by acquiring the symptom information of the patient and are sent to the doctor as reference cases, such that a lot of inquiring time can be saved. Furthermore, the doctor can adopt a definitive therapy by means of the reference cases, such that the treatment efficiency of the patient is higher.

### BRIEF DESCRIPTION OF THE DRAWINGS

In order to describe the technical schemes in the embodiments of the disclosure more clearly, brief introduction on drawings needed to be used in the embodiments will be made below. It is obvious that the drawings described below are merely some embodiments of the disclosure, and those skilled in the technical field further can obtain other drawings according to the drawings without creative efforts.

FIG. 1 is a flow diagram of the medical data processing method provided by the embodiment of the application.

### DETAILED DESCRIPTION

Further description of specific embodiments of the present invention in detail will be made below in combination with drawings. It should be noted herein that description on these embodiments is used for assisting understanding of the present invention and does not constitute limitation of the present invention. In addition, technical features involved in the described embodiments of the present invention can be combined with one another as long as they do not conflict with each other.

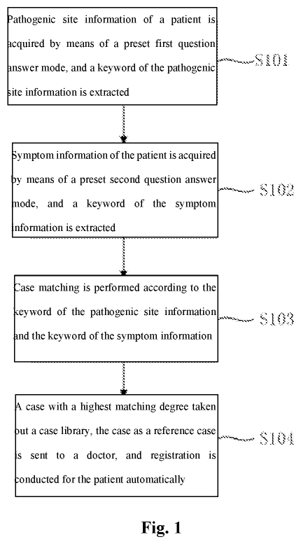

Referring to FIG. 1, a flow diagram of the medical data processing method provided by the embodiment is illustrated, the method including;

S101, pathogenic site information of a patient is acquired by means of a preset first question answer mode, and a keyword of the pathogenic site information is extracted;

In an embodiment, after acquiring pathogenic site information of a patient by means of a preset first question answer mode, and extracting a keyword of the pathogenic site information, the method further includes:
a pathogenic site of the patient is acquired according to the keyword of the pathogenic site information.

The first question answer mode can be a question answer rule formulated by a physician or a specialist according to inquiring experience.

S102, symptom information of the patient is acquired by means of a preset second question answer mode, and a keyword of the symptom information is extracted.

In an embodiment, before acquiring symptom information of the patient by means of a preset second question answer mode, and extracting a keyword of the symptom information, the method further includes:
a corresponding second question answer mode is selected according to the pathogenic site of the patient.

As the pathogenic symptoms of different pathogenic conditions are different, in order to acquire the pathogenic conditions of the patient in a targeted manner, it is necessary to confirm the pathogenic site of the patient first and select the corresponding second question answer mode according to the pathogenic site. The second question answer mode can be one of the preset question answer modes. The preset second question answer mode can be a question answer rule formulated by a physician or a specialist according to inquiring experience.

If the pathogenic site is head, a head disease question answer mode is selected automatically. Specific problems to be inquired can be as follows: whether the patient has a headache or not, whether the patient suffers from insomnia or not, how about a pathogenic period, whether the patient has other syndromes or not and the like.

If the pathogenic site is heart, a heart disease question answer mode is selected automatically. Specific problems to be inquired can be as follows: whether the patient has a high blood pressure history or not, whether the patient suffers from palpitation or not, whether the patient suffers from angina or not, whether the patient has other syndromes or not and the like.

By inquiring the pathogenic conditions of the patient by the preset question answer modes, the type of disease of the patient can be determined quickly, and a condition of waste of medical resource and money as a user registering selects a wrong department can be further avoided.

In an embodiment, extracting a keyword of the pathogenic site information includes:
the symptom information is extracted according to the preset keyword extraction mode.

Different second question answer modes correspond to different keyword extraction modes. After the pathogenic site of the patient is acquired, the corresponding pathogenic condition question answer mode and keyword extraction modes can be selected to acquire corresponding symptom keywords, and each question answer mode corresponds to one keyword extraction mode, such that precision of extracting the symptom information is guaranteed.

S103, case matching is performed according to the keyword of the pathogenic site information and the keyword of the symptom information.

The case is classified according to the keyword of the acquired pathogenic site information, the pathogenic site of the patient is determined, and then the case is matched with the case in the case library according to the symptom keyword.

In an embodiment, before performing case matching according to the symptom keyword, the method further includes:
the case in an existing case library is classified according to the pathogenic site.

After classifying the case in an existing case library according to the pathogenic site, the method further includes:

Keywords of different cases are acquired and the keywords are taken as case tags and are stored.

When case matching is performed according to the keyword of the pathogenic site and the keyword of the symptom information, it is only necessary to inquire tag information of the case to obtain a matching result, such that a lot of time and resource are saved. The case corresponding to the tag matching with the most keywords can be considered as the case with a highest matching degree.

S104, a case with a highest matching degree taken out a case library, the case as a reference case is sent to a doctor, and registration is conducted for the patient automatically.

In an embodiment, the case corresponding to the tag with a highest matching degree with the symptom keyword of the patient is extracted from the case library and is sent to the doctor of the corresponding department as the reference case, registration is conducted on the patient automatically, and the doctor conducts diagnosis on the patient according to the reference case, such that a lot of time can be saved. The doctor further can conduct therapy on the patient in a targeted manner according to medication and treatment conditions of the reference case. Further, after the case is sent to the doctor, registration is conducted on the patient automatically, such that a condition of waste of money and time as the patient misjudges the pathogenic condition and conducts registration mistakenly can be avoided.

Further description of specific embodiments of the present invention in detail will be made above in combination with drawings. But the present invention is not limited to the described embodiments. Various changes, modifications, substitutions and alterations made to these embodiments by those skilled in the art without departing from the principle and the spirit of the present invention still fall within the protection scope of the present invention.

1. A medical data processing method, comprising:
acquiring pathogenic site information of a patient by means of a preset first question answer mode and extracting one or more keywords of the pathogenic site information;
acquiring symptom information of the patient by means of a preset second question answer mode and extracting one or more keywords of the symptom information;
performing case matching according to the one or more keywords of the pathogenic site information and the one or more keywords of the symptom information; and
taking out a case, or cases, with a highest matching degree from a case library, wherein the highest matching degree is calculated according to the one or more keywords of the pathogenic site information and the one or more keywords of the symptom information, sending the case or cases as a reference case to a doctor, and conducting registration for the patient automatically.

2. The medical data processing method as claimed in claim 1, wherein after extracting a keyword of the pathogenic site information, the method further comprises:
acquiring a pathogenic site of the patient according to the keyword of the pathogenic site information.

3. The medical data processing method as claimed in claim 2, wherein before acquiring symptom information of the patient by means of a preset second question answer mode and extracting a keyword of the symptom information, the method further comprises:
selecting a corresponding second question answer mode according to the pathogenic site of the patient to acquire other pathogenic site information of a patient.

4. The medical data processing method as claimed in claim 1, wherein before matching the keyword with a case in the case library, the method further comprises:
classifying the case in the case library according to the pathogenic site and extracting a pathogenic symptom keyword as a case tag and storing the pathogenic symptom keyword.

5. The medical data processing method as claimed in claim 4, wherein the taking out a case with a highest matching degree from a case library comprises:
matching the keyword of the symptom information with the case tag and taking out the case corresponding to the case tag with the highest matching degree from the case library.

6. The medical data processing method as claimed in claim 1, wherein the preset first question answer mode and the preset second question answer mode are question answer rules formulated by a physician or a specialist.

7. A medical data processing system, comprising:
a first question answer module configured to acquire pathogenic site information of a patient by means of a preset first question answer mode and extract one or more keywords of the pathogenic site information;
a second question answer module configured to acquire symptom information of the patient by means of a preset second question answer mode and extract one or more keywords of the symptom information;
a matching module configured to perform case matching according to the one or more keywords of the pathogenic site information and the one or more keywords of the symptom information; and
a case acquisition module configured to take out a case or cases with a highest matching degree from a case library, wherein the highest matching degree is calculated according to the one or more keywords of the pathogenic site information and the one or more keywords of the symptom information, and send the case or cases as a reference case to a doctor, and conduct registration for the patient automatically.

8. (canceled)

9. (canceled)
